(19) United States
(12) Patent Application Publication
(10) Pub. No.: US 2009/0246882 A1
(43) Pub. Date: Oct. 1, 2009

(54) System for Measurement of Dissolved Organic Compounds in Water

(76) Inventors: Rocco D. Pochy, Fremont, CA (US); Scott S. Salton, Fremont, CA (US); Thomas C. Saunders, Fremont, CA (US)

Correspondence Address: GREENBERG & LIEBERMAN, LLC, 2141 WISCONSIN AVE, N.W., SUITE C-2, WASHINGTON, DC, 20007, US

(21) Appl. No.: 12/416,773

(22) Filed: Apr. 1, 2009

Related U.S. Application Data
(60) Provisional application No. 61/041,498, filed on Apr. 1, 2008.

(51) Int. Cl. G01N 33/00

(52) U.S. Cl. 436/146

(57) ABSTRACT

A system for measuring dissolved organic compounds in water that engages separate conductivity and temperature sensors at various points in the water flow. In addition, a UV reaction chamber produces light that levies high-level amounts of hydroxyl radicals during the oxidation process. The flow is then diverted into various directions based upon the settings of a three-way valve that determines when the sensor readings will take place as the water flows through the UV radiation.

This is a non-provisional application claiming priority to provisional patent application No. 61/041,498 filed on Apr. 1, 2008.

### FIELD OF THE INVENTION

The present invention is a system for measuring dissolved organic compounds; and more particularly, the present invention employs a UV reaction chamber to purify water and then sample the water to ensure that a desired level of removal of organic compounds has been achieved.

### BACKGROUND OF THE INVENTION

Water purity is crucial in many applications. In fact, there is even a definition of that which can be called “purified” water versus “filtered” water. A fundamental indicator relating to the purity of the water is based on the measurement of organic compounds that are dissolved in the water.

Typically, the measurement of dissolved organic compounds is normally conducted via a total organic carbon (TOC) analyzer. This normally works by breaking down the carbon compounds to carbon dioxide, which reacts with the water to form carbolic acid. At this point in the traditional process, the conductivity of the solution is changed. By measuring the conductivity and temperature of the difference between the start and end of the oxidation process, a user can calculate the amount of carbon converted into carbon dioxide.

The use of common ultra violet (UV) sources such as mercury lamps requires extended exposure times to complete the oxidation process. The use of reagents is sometimes used as a catalyst to speed up this reaction. However, this scenario requires the user to constantly monitor and maintain a supply of reagents to assure operation of the apparatus. It also should be noted that various TOC values that are not immediately detected could detrimentally affect the safety and contamination levels of products. Because of these issues relating to the important area of water purity, there is a need for an apparatus that can perform rapid oxidation without the need for catalysts or reagents.

The present invention solves this need in a novel manner. Through the use of a highly efficient UV reaction chamber, the present invention performs the rapid oxidation of carbon compounds without the need for catalysts or reagents. Moreover, the present invention minimizes contamination by limiting contact with surfaces that are prone to contamination. The present invention also solves the TOC problems by detecting TOC values rapidly for improved safety, prevention of damage to products by contamination, and better control of the processes.

### SUMMARY OF THE PRESENT INVENTION

The present invention is an apparatus that serves to perform rapid oxidation of carbon compounds while at the same time, reduces the prospects for contamination. The purpose of these functions is to measure the dissolved organic compounds in water to provide meaningful indicators relating to the purity of the water.

The present invention begins operation as water passes through a filter with a bypass that serves to filter out any bubbles contained in the water. These bubbles are gravity fed to a bypass that leads to the outlet of the present invention. In the preferred embodiment of the present invention, the filter will be a 100-micron filter. The flow is then regulated by the flow controller, where the water ultimately passes through a conductivity and temperature sensor. The conductivity and temperature sensor records the initial conditions of the fluid.

From there, the water runs into the UV reaction chamber, which is a fundamental element of the present invention. When the water runs into the UV reaction chamber, the water is exposed to intense UV radiation where the organic compounds are broken down. A three-way valve causes the flow to be directly moved to a flow meter or can be diverted instead through a second conductivity and temperature sensor for a second reading. Based on the readings gleaned from G1, T1 sensors and G0,T0 sensors, the amount of carbon present in the water can be calculated. The water then exits the apparatus via the outlet.

The present invention also features two modes in the preferred embodiment. These modes are referred to as the light mode and the dark mode. The system of the present invention oscillates between the two modes to provide periodic reads of the water flowing through the system.

### BRIEF DESCRIPTION OF THE DRAWINGS

FIG. 1 is a view of the TOC analysis system of the present invention.

FIG. 2 is a view of the UV reaction chamber.

FIG. 3 is a view of the light mode of the present invention.

FIG. 4 is a view of the dark mode of the present invention.

### DETAILED DESCRIPTION OF THE DRAWINGS

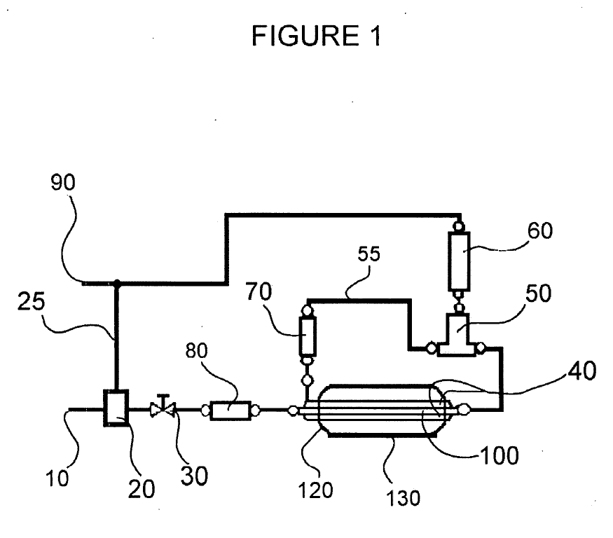

In FIG. 1, we see a view of the system of the present invention that highlights the various elements relating to the water flow through the TOC analysis system. As we see in FIG. 1, a sample inlet (10) is where the water sample initially enters the system of the present invention. The flow of the water is pushed through the system via conventional means. Once the water enters the system via the sample inlet (10), the incoming water passes through a filter (20). The preferred embodiment of the present invention cites the filter (20) at 100 microns. At this point of the filter (20) serves as a conduit to the bypass (25) where any bubbles contained in the water are filtered out of the water sample. The bubbles are gravity fed through the bypass (25) where the bubbles are ultimately released from the system at the sample outlet (90). As the bubbles are filtered out of the water sample, the water continues to flow through the system.

The first point in the system to greet the flow is a flow regulator (30), which regulates the flow of the water in such aspects as speed and control. With the flow under control via the flow regulator (30), the water then passes through the G1,T1 sensors (80) of the present invention. The G1,T1 sensors (80) are conductivity and temperature sensors that are comprised of cells in the preferred embodiment. The G1,T1 sensors (80) record the initial conditions of the fluid.

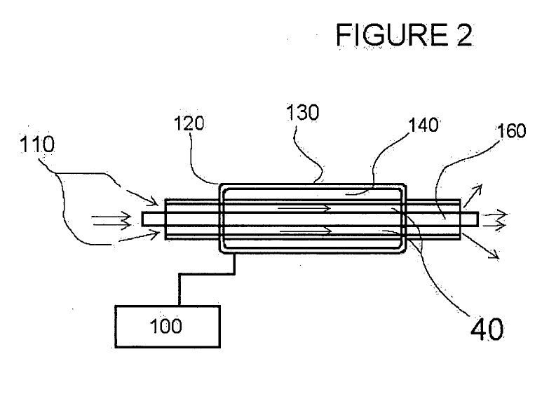

From this point, the water then flows into the UV reaction chamber (40). The UV reaction chamber (40) is better viewed in FIG. 2. The UV reaction chamber (40) serves to expose the water that has flowed into it to intense UV radiation where the organic compounds of the water is broken down. As we see in FIG. 2, the water enters the UV reaction chamber (40) at the fluid intake (110). In the preferred embodiment of the present invention, the UV reaction chamber (40) is enclosed by a quartz reactor (120). The quartz reactor (120) is a thin layer of high-purity fused quartz that caters to very high UV transmission. The purpose of the quartz reactor (120) is to allow for extremely low loses and simplified construction. The quartz reactor (120) also solidifies the process because it does not leave any gaps, meaning that UV radiation is prevented from being lost due to reflection and absorption.

The UV reaction chamber (40) also is enclosed by a metallic coating (130) in the preferred embodiment. The metal coating (130) is applied to the outer shell of the discharge gas element (140) to act as an electrode. The UV reaction chamber (40) itself in the preferred embodiment produces light at wavelengths of 160 nm to 190 nm. These confines in respect to light lead to high-level production of hydroxyl radicals, which are beneficial to organic oxidation.

Once the water flow passes the UV reaction chamber and the organic compound is broken down, the flow reaches a three-way valve (50) as seen in FIG. 1. As the flow enters into the three-way valve (50), the flow is directed to either a flow meter (60) or can pass through a diversion (55) that leads the flow to the G0,T0 sensor (70). The G0,T0 sensor (70) is a second conductivity and temperature sensor that conducts a second reading of the water sample. Based on the reading through the use of the G0,T0 sensor (70), combined with the previous G1,T1 sensor (80), the amount of carbon present in the water can be calculated. The water then is pushed through the system until it ultimately exits at the sample outlet (90). It should be noted that the G1,T1 sensor (80) and the G0,T0 sensor (70) are separate from the UV reaction chamber (40) because by separating these elements, it prevents interactions between the UV radiation and the various sensors that could otherwise skew the readings or cause bubble formations on the surface.

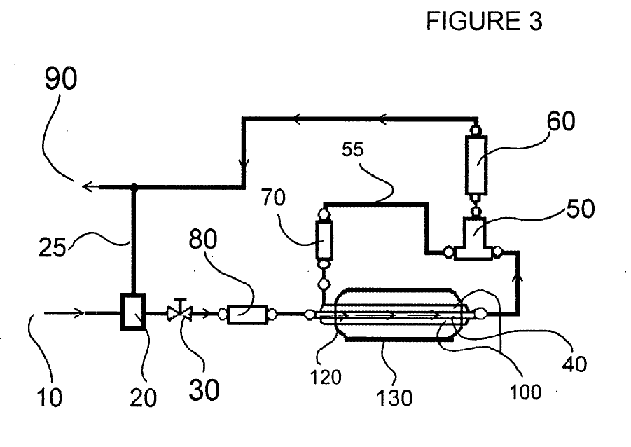

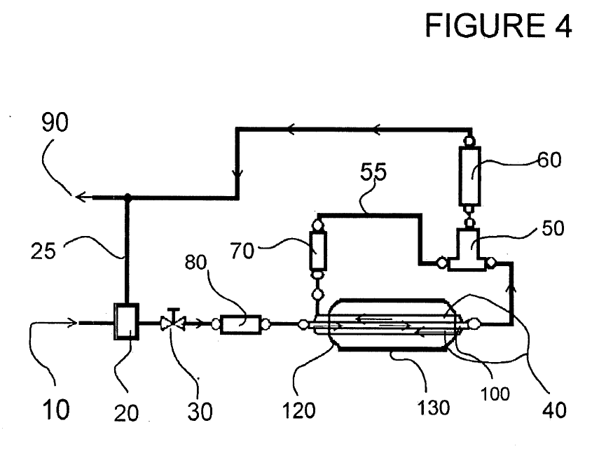

As we see in FIG. 3 and FIG. 4, the preferred embodiment utilizes two modes relating to the cycle of flow through the system. The two modes are referred to as the light mode of FIG. 3 and the dark mode of FIG. 4. The modes operate via settings of the three-way valve (50).

The light mode as seen in FIG. 3 relates to water flowing through the system. The arrows of FIG. 3 depict the direction of flow. The three-way valve (50) is set to prevent water from passing through the G0,T0 sensors (70). This prevention from passing water through the diversion (55) results in the fact that the water is trapped in a layer between the coolant tube (160) and the UV reaction chamber (40). In the preferred embodiment, high voltage emitting from the high voltage power supply (100) is applied to the UV reaction chamber (40). This causes the discharge gas in the discharge gas element (140) to fluoresce with UV radiation. Based on calibrated time values, the UV light remains on to fully oxidize the organic compounds present in the solution. During this time, water is continually running through the coolant tube (160) in order to minimize excessive heating of the water being exposed to the radiation.

In FIG. 4, we see the dark mode of the present invention. The dark mode is represented in FIG. 4 by arrows that depict the cycle of water flow for this aspect of the system. The dark mode of FIG. 4 occurs after the UV exposure has been completed. In the dark mode, the three-way valve (50) switches in order to prevent the flow of water to go directly to the flow meter (60). Instead, the three-way valve (50) pushes the water out of the UV reaction chamber (40) and forces it through the G0,T0 sensors (70). In FIG. 4 we see that the arrows represent the flow of the water. In the preferred embodiment, the coolant tube (160) will have a bypass hole that will be the conduit for the water to be forced out.

As the water in the dark mode passes through the G0,T0 sensors (70), the appropriate conductivity and temperatures are recorded. This process fills the UV reaction chamber (40) with fresh water which is ready to be oxidized with the system and is switched back into the light mode. In fact, the system of the present invention oscillates between the light mode and the dark mode. This oscillation provides periodic reads of the water flowing through the system.

We claim:

1. A system of measurement of dissolved organic compounds in water, comprising:
passing a water sample through a sample inlet and pushing the water sample through a filter, the filter serving as a conduit to a bypass where any bubbles contained in the water sample are filtered out of the water sample;
feeding the bubbles via gravity through the bypass such that the bubbles are ultimately released at a sample outlet;
greeting a flow of the water sample at a first point, the first point being a flow regulator;
regulating speed and control of the flow of the water sample via the flow regulator;
passing the water sample through G1,T1 sensors after the water sample passes the flow regulator;
recording initial conditions of the water sample via the G1,T1 sensors, the G1,T1 sensors sensing conductivity and temperature;
flowing the water sample into a UV reaction chamber after the water sample passes through the G1,T1 sensors;
exposing the water sample flowing into the UV reaction chamber with intense UV radiation such that organic compounds in the water sample are broken down;
entering the water sample into the UV reaction chamber at a fluid intake;
enclosing the UV reaction chamber in a quartz reactor, the quartz reactor being a thin layer of high-purity fused quartz;
enclosing the UV reaction chamber in a metallic coating, the metallic coating applied to an outer shell of a discharge gas element to act as an electrode;
producing light via the UV reaction chamber at wavelengths of 160 nm to 190 nm;
passing the flow of the water sample into a three-way valve after the water sample passes the UV reaction chamber and organic compounds are broken down;
directing the flow via the three-way valve to either a flow meter or a diversion leading the flow to a G0,T0 sensor;
conducting a second reading of the water sample at the G0,T0 sensor, the G0,T0 sensor being a second conductivity and temperature sensor.
calculating an amount of carbon present in the water sample based on a reading through use of the G0,T0 sensor combined with a previous reading of the G1,T1 sensor;
pushing the water sample to an exit at a sample outlet;
separating the G1,T1 sensor and the G0,T0 sensor from the UV reaction chamber to prevent the skewing of readings and bubble formations on a surface;
utilizing a light mode and a dark mode relating to cycles of the flow of the water sample, the light mode and the dark mode operating via settings of the three-way valve;
preventing the water sample from passing through the G0,T0 sensors and instead into the diversion via the three-way valve when set to the light mode;
applying high voltage from a high voltage power supply to the UV reaction chamber when in the light mode;
oxidizing the organic compounds via having UV light based on calibrating time values in the light mode while at the same time, continually running the water sample through a coolant tube;
switching the three-way valve in order to prevent the flow of the water sample from going directly to the flow meter while in the dark mode;
pushing the water sample via the three-way valve out of the UV reaction chamber and through the G0,T0 sensors while in dark mode;
recording conductivity and temperatures as the water sample in the dark mode passes through the G0,T0 sensors;
filling the UV reaction chamber with fresh water which is ready to be oxidized during the dark mode such that the dark mode can then be switched back into the light mode; and
oscillating between the dark mode and the light mode.

2. The system of claim 1, further comprising citing the filter at 100 microns.

3. The system of claim 1, further comprising pushing the water sample through as the bubbles are filtered out of the water sample.

4. The system of claim 1, further comprising preventing UV radiation from being lost due to reflection and absorption via the quartz reactor.

5. The system of claim 4, further comprising preventing gaps within the quartz reactor.

6. The system of claim 1, further comprising producing a high-level amount of hydroxyl radicals via production of light at wavelengths of 160 nm to 190 nm.

7. The system of claim 1, further comprising trapping the water sample in a layer between the coolant tube and the UV reaction chamber when in the light mode.

8. The system of claim 1, further comprising causing a discharge of gas in a discharge gas element to fluoresce with UV radiation when applying high voltage from a high voltage power supply to the UV reaction chamber when in the light mode.

9. The system of claim 1, further comprising minimizing excessive heating of the water sample being exposed to UV radiation by continually running the water sample through a coolant tube.

10. The system of claim 1, further comprising providing a conduit for the water sample to be forced out while in the dark mode via a bypass hole formed with the coolant tube.

11. The system of claim 1, further comprising providing periodic reads of the water sample via the oscillation between the dark mode and the light mode.
